(19) United States
(12) Patent Application Publication
(10) Pub. No.: US 2004/0248287 A1
(43) Pub. Date: Dec. 9, 2004

(54) Multi-array systems and methods of use thereof

(76) Inventors: Qianjin Hu, Castro Valley, CA (US); Allan Peng, Palo Alto, CA (US); James Robert Love JR., Millbrae, CA (US)

Correspondence Address: MORRISON & FOERSTER LLP, 755 PAGE MILL RD, PALO ALTO, CA, 94304-1018, US

(21) Appl. No.: 10/810,258

(22) Filed: Mar. 26, 2004

Related U.S. Application Data
(60) Provisional application No. 60/458,911, filed on Mar. 28, 2003.

(51) Int. Cl. C12Q 1/68; C12M 1/34

(52) U.S. Cl. 435/287.2; 435/6

(57) ABSTRACT

Multi-array systems useful for performing assays on multiple biomolecule arrays simultaneously while minimizing sample volume are provided. Apparatuses and kits comprising the multi-array systems also provided. Methods of using the multi-array systems in assays such as hybridization arrays are also provided, as well as methods of producing the multi-array systems.

### RELATED INVENTIONS

This application claims the priority benefit of U.S. Provisional Application No. 60/458,911, filed Mar. 28, 2003, the contents of which are hereby incorporated by reference into the present disclosure in their entirety.

### FIELD OF THE INVENTION

This invention relates generally to the field of microarray assays. More specifically, the invention provides apparatuses and methods for performing assays on multiple microarrays simultaneously while minimizing sample volume.

### BACKGROUND OF THE INVENTION

A wide variety of molecular biological techniques involving analysis of nucleic acids and proteins form the basis of clinical diagnostics and important research tools. These techniques include nucleic acid hybridization and genetic sequence analysis and often require carrying out numerous operations on a large number of samples (see, e.g., Sambrook, J., et al., Molecular Cloning: A Laboratory Manual. Cold Spring Harbor Laboratory Press, Cold Spring Harbor, N.Y. (2nd ed. 1989)). In addition, current trends in medical diagnostic testing and pharmaceutical research require conducting a large number of tests concurrently on a single device.

Nucleic acid hybridization generally involves the detection of small numbers of target nucleic acids (DNA and RNA) among a large amount of non-target nucleic acids with a high degree of specificity. Stringent hybridization conditions are necessary to maintain the required degree of specificity and various combinations of agents and conditions such as salt, temperature, solvents, denaturants and detergents are used for the purpose. Nucleic acid hybridization has been conducted on a variety of solid support formats (see, e.g., Beltz, G. A., et al., Methods in Enzymology, Vol. 100, part B, 19: 266-308, Academic Press, NY (1985)).

Recent developments in DNA microarray technology make it possible to conduct a large-scale assay of a plurality of target molecules on a single solid phase support. Generally, a DNA chip including an oligonucleotide array is comprised of a number of individual oligonucleotides linked to a solid support in a regular pattern such that each oligonucleotide is positioned at a known location. After generation of the array, samples containing the target sequences are exposed to the array, hybridized to the complementing oligonucleotides bound to the array, and detected using a wide variety of methods, most commonly radioactive or fluorescent labels. U.S. Pat. No. 5,837,832 (Chee et al.) and related patent applications describe immobilizing an array of oligonucleotide probes for hybridization and detection of specific nucleic acid sequences in a sample.

Microarray analysis is also useful for analysis of proteins. In some embodiments, assays using protein microarrays involve the use of an array of antibodies to analyze for the presence and/or quantity of proteins in a solution sample.

Limitations of microarray analysis of both proteins and nucleic acids include the difficulty of detecting nucleic acids that are available only in small volumes and small quantities. Large numbers of compounds need to be presented within reasonably sized reaction volumes. Since most test samples are of biological origin, they are typically very expensive, difficult to prepare and in short supply. Examples of test samples are PCR products or purified drug receptors, which are typically available in microliter quantities. In most cases, DNA synthesis requires the use of expensive components, such as in phosphoramidite DNA synthesis, so that the surface area of the array is also important during its manufacture.

In addition, some applications of microarray analysis require that extremely dense arrays be exposed to samples which are typically of very limited quantity. For instance, the simultaneous testing of a sample against the entire human genome requires the exposure of the sample to a microarray(s) containing no less than about 40,000-50,000 features (different oligonucleotides). Also, applications such as universal arrays and DNA sequencing on a chip require microarrays with a maximum number of features on the array in order to be successful. As the complexity and size of the microarrays increases, so does the demand on sample volume.

Although decreasing the size of the array elements is helpful in reducing the cost of using the arrays, size limitations still exist. Small feature sizes can complicate manufacturing and detection processes. Thus, there remains a need to develop more effective ways for minimizing sample volume requirements of microarray assays.

### BRIEF SUMMARY OF THE INVENTION

The present invention provides novel devices and methods for performing assays on multiple biomolecule arrays simultaneously while minimizing the sample volume used.

In one aspect, the invention provides a multi-array system comprising the following: (a) a first solid substrate having a first surface; (b) a second solid substrate having a second surface, wherein the first and second solid substrates are positioned so that the first surface faces the second surface; (c) a spacer contacting and separating the first and second solid substrates, so as to form at least one reaction chamber comprising a fluid-receiving space between the first and second surfaces; (d) a first biomolecule array immobilized on the first surface; and (e) a second biomolecule array immobilized on the second surface, wherein the first and second biomolecule arrays are exposed to the fluid-receiving space.

In some embodiments, the reaction chamber is substantially enclosed by the first and second surfaces and the spacer. In some embodiments, the reaction chamber comprises at least one opening.

In some embodiments, the multi-array system further comprises fluid in the fluid-receiving space of the reaction chamber, such that the fluid is in contact with the first and second biomolecule arrays.

In some embodiments, each of the first and second surfaces is substantially planar. In some embodiments, the first and second surfaces are in substantially parallel planes.

In some embodiments, the spacer is removably adhered to the first and/or second surface.

In some embodiments, the spacer is made of plastic, rubber or Teflon®. In some embodiments, the material from which the spacer is made comprises silicone rubber.

In another aspect, the invention provides an apparatus comprising a multi-array system described herein and a temperature control unit, wherein the temperature control unit functions (i.e., operates) to alter the temperature of the reaction chamber. For instance, in some embodiments, the apparatus comprises a thermal cycler, a water bath, or an air system (such as a hybridization oven).

In another aspect, the invention provides an apparatus comprising a multi-array system described herein and further comprising a thermal cycler that has a first temperature block in thermal contact with the first solid substrate of the multi-array system. In some embodiments, the thermal cycler of the apparatus further comprises a second temperature block, the second temperature block being in thermal contact with the second solid substrate of the multi-array system.

In another aspect, the invention provides an article of manufacture such as a kit for use in performing an assay that comprises a multi-array system described herein and an assay solution or a solution for use in performing an assay with the multi-array system.

In another aspect, the invention provides an article of manufacture such as a kit for use in performing an assay that comprises the multi-array system described herein and instructions regarding the use of the multi-array system in at least one step of an assay.

In yet another aspect, the invention provides a method of performing an assay, comprising using a multi-array system, apparatus, and/or kit described herein in at least one step of the assay.

In another aspect, the invention provides a method of performing an assay on a plurality of biomolecule arrays (i.e., two or more biomolecule arrays) simultaneously, where the method comprises the step of performing the assay using a multi-array system, apparatus, and/or kit described herein.

In still another aspect, the invention provides a method of making a multi-array system, the method comprising the following steps: (a) providing a first substrate having a first surface on which a first biomolecule array is immobilized; (b) providing a second solid substrate having a second surface on which a second biomolecule array is immobilized; and (c) fixably positioning the first and second solid substrates using a spacer so as to form a reaction chamber in which the first and second surfaces face each other and are separated by a fluid-receiving space and in which the first and second biomolecule arrays are exposed to the fluid-receiving space.

### BRIEF DESCRIPTION OF THE DRAWINGS

FIG. 1 shows top (1A), side (1B), and front (1C) views of glass slides used in some embodiments of a double-array system.

FIG. 2 shows top (2A), side (2B), and front (2C) views of a U-shaped gasket used in one embodiment of a double-array system.

FIG. 3 shows top (3A), side (3B), and front (3C) views of a side shim used in one embodiment of a double-array system.

FIG. 4 shows side (4A), bottom (4B), and front (4C) views of a bottom shim used in one embodiment of a double-array system.

FIG. 5 shows top (5A), side (5B), and front (5C) views of a side shim used in one embodiment of a double-array system.

FIG. 6 shows top (6A), side (6B), and front (6C) views of one embodiment of a double-array system comprising the use of a U-shaped gasket as a spacer.

FIG. 7 shows a detailed top view of the double-array system of FIG. 6.

FIG. 8 shows top (8A), side (8B), and front (8C) views of an embodiment of the double-array system of FIGS. 6 and 7 around which shims have been placed.

FIG. 9 shows a side (9A) and front (9B) view of one embodiment of a double-array system positioned between two thermal blocks of a thermal cycler. FIG. 9C shows a top elevational edge view taken along the line 9C-9C of FIG. 9B. (The remainder of the thermal cycler is not shown in the figure.)

FIG. 10 shows another embodiment of the double-array system in which a plastic holder is used as a spacer. FIG. 10A shows the front view of the double-array system. FIG. 10B shows a cross-sectional view of the double-array system of FIG. 10A taken along the line 10B-10B of FIG. 10A, although two temperature blocks of a thermal cycler are additionally illustrated sandwiching the double-array system. (The remainder of the thermal cycler is not shown.)

FIG. 11 shows another embodiment of the invention, a double-array system comprising a rectangular-shaped rubber gasket. FIGS. 11A and 11B show top views of some of the components of the double-array system shown in a perspective view in FIG. 11C. FIG. 11D shows a cross-sectional view of the double-array system taken along the line 11D-11D of FIG. 11C. FIG. 11E shows a cross-sectional front view of the double-array system taken along the line 11E-11E of FIG. 11C.

FIG. 12 shows a cross-sectional front view of the double-array system of FIG. 11C into which syringe needles have been inserted to facilitate the transfer of fluid into the reaction chamber.

### DETAILED DESCRIPTION OF THE INVENTION

The present invention provides devices useful for decreasing the sample volumes required to perform microarray assays, and methods of using those devices. The devices are also useful in that they allow for the simultaneous exposure of multiple arrays to a given sample solution. For instance, in one embodiment, the multi-array system is a double-array system comprising two microarrays, each of which comprises 20,000-25,000 different oligonucleotides corresponding to portions of human gene sequences, wherein the double-array system can be used to survey the entire human genome simultaneously in a single hybridization reaction requiring about half the sample volume required to perform the same hybridization reaction on the two microarrays separately.

In one aspect, the invention provides a multi-array system comprising the following: (a) a first solid substrate having a first surface; (b) a second solid substrate having a second surface, wherein the first and second solid substrates are positioned so that the first surface faces the second surface; (c) a spacer contacting and separating the first and second solid substrates, so as to form at least one reaction chamber comprising a fluid-receiving space between the first and second surfaces; (d) a first biomolecule array immobilized on the first surface; and (d) a second biomolecule array immobilized on the second surface, wherein the first and second biomolecule arrays are exposed to the fluid-receiving space.

In some embodiments, the multi-array system comprises a total of two solid substrates on which are immobilized a total of two biomolecule arrays (one on each substrate). However, in some embodiments, the multi-array system comprises additional solid substrates bearing additional biomolecule arrays. For instance, in some embodiments, the multi-array system comprises three or four or more substrates bearing biomolecule arrays.

In addition, in some embodiments, the multi-array system comprises a first solid substrate comprising more than one biomolecule array on its surface and/or a second solid substrate comprising more than one biomolecule array on its surface.

In some embodiments, the solid substrate and its surface form a rigid support on which to carry out the reactions described herein. The substrate and its surface are also chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, polyethylene, polypropylene, polyvinyl chloride, poly(methyl acrylate), poly(methyl methacrylate), or combinations thereof. In some embodiments, each of the first and second solid substrates comprises a material selected from the group consisting of glass, plastic, and silicon. Other substrate materials will be readily apparent to those of ordinary skill in the art upon review of this disclosure. In some embodiments, the substrate is flat glass or single-crystal silicon. In some embodiments each of the first and second substrates is a glass slide (of any size and/or shape).

Surfaces on the solid substrate will usually, though not always, be composed of the same material as the substrate. Thus, the surface may be composed of any of a wide variety of materials, for example, polymers, plastics, ceramics, polysaccharides, silica or silica-based materials, carbon, metals, inorganic glasses, membranes, or composites thereof. The surface is functionalized with binding members which are attached firmly to the surface of the substrate. In some embodiments, the surface functionalities will be reactive groups such as silanol, olefin, amino, hydroxyl, aldehyde, keto, halo, acyl halide, or carboxyl groups. In some cases, such functionalities preexist on the substrate. For example, silica based materials have silanol groups, polysaccharides have hydroxyl groups, and synthetic polymers can contain a broad range of functional groups, depending on which monomers they are produced from. Alternatively, if the substrate does not contain the desired functional groups, such groups can be coupled onto the substrate in one or more steps.

In some embodiments, the solid substrates are coated. For instance, in some embodiments the solid substrates are glass slides (of any size or shape) that are coated. In some embodiments, the glass slides are coated with a three-dimensional surface chemistry comprised of a long-chain, hydrophilic polymer (such as polyacrylic polymers) containing amine-reactive groups. This polymer is covalently crosslinked to itself and to the surface of the slide. The cross-linked polymer, in combination with end-point attachment, orients the immobilized DNA, and holds it away from the surface of the slide. Additionally, the hydrophilic nature of the polymer provides a passivating effect once the DNA has been immobilized resulting in lower background. Examples of such slides include CodeLink™ Activated Slides (Amersham Biosciences, Piscataway, N.J.) or 3D-Link™ slides (SurModics, Inc., Eden Prairie, Minn.). These slides are coated with a hydrophilic polymer containing N-hydroxysuccinimide (NHS) ester reactive groups. Some examples of methods and reagents for covalent attachment of nucleic acids onto a substrate are described in U.S. Pat. No. 6,465,178 (Chappa, et al.).

In some embodiments, glass slides are coated with materials such as aminosilane for non-covalent binding of nucleic acids. In some embodiments, the coating material also comprises linkers (e.g., isothiocyanate linkers) for covalent attachment to amino acids (Genorama™ Microarray Slides, Sunergia Group, Reston, Va.).

In some embodiments, the first and second solid substrates are coated glass slides or nylon-overlaid glass slides. (The glass slides may be of any size or shape. For instance, some glass slides have dimensions of about 25 mm by about 75 mm. However, glass slides of different dimensions such as about 2 inches by about 3 inches are also contemplated.) In some embodiments, the first and second solid substrates are both nitrocellulose-film glass slides (e.g., from Schleicher & Schuell, Keene, N.H.). In some embodiments, the solid substrates are nylon-overlaid glass slides. In some of these embodiments, the substrates have thin uniform membranes, such as nylon membranes, adhered to a glass slide for binding with no necessity for specialized cross-linking reagents (e.g., Vivid™ Gene Array Slides, Pall Corp., East Hills, N.Y.). The nylon membrane surface strongly binds both cDNA's and oligonucleotides and exhibits stronger signals than traditional glass slides.

The size of the solid substrate can be of any size and shape. In some embodiments, each of the first and second surfaces of the substrates of the multi-array system is substantially planar. In some embodiments, the substantially planar surface is a planar surface. In some embodiments, substantially planar surfaces are those surfaces comprising surface features such as wells, channels, posts and the like, which are small in comparison to the dimensions of the solid substrate surface itself. In other words, in some embodiments, the features of the surfaces are primarily two-dimensional, rather than three-dimensional in nature. (For instance, in some embodiments, the height of a surface feature is less than about 1/100 or less than about 1/1000 of the length and width dimensions of the surface.) In some embodiments, the first and second surfaces are each planar. In some embodiments, the configuration of each of the first and second surfaces is rectangular, square, or circular.

In some embodiments, the first and second solid substrates of the multi-array system each have two substantially planar or planar sides. For instance, in some embodiments, each of the first and second solid substrates is a glass slide. In some embodiments, each of the first and second solid substrates is a glass slide measuring approximately 76 mm long by approximately 25.5 mm wide by approximately 0.5 mm thick.

In some embodiments, the first and second surfaces of the multi-array system are in substantially parallel planes. In some embodiments, the first and second surfaces are in planes that are parallel to each other.

The multi-array systems of the present invention comprise at least two microarrays. A “microarray” is an array of preferably discrete regions, each having a defined area, formed on the surface of a solid substrate. In some embodiments, the microarray is a linear or two-dimensional array of the preferably discrete regions or “spots.” In some embodiments, the total area of the microarray is less than about 400 cm². A “biomolecule array” or “array of biomolecules” is a microarray in which the regions comprise immobilized biomolecules. In some embodiments, the total area of the biomolecule array is less than about 400 cm², or less than about 100 cm², or less than about 25 cm², or less than about 10 cm². In some embodiments, the area of the biomolecule array is less than about 1 cm². In some embodiments, the density of the discrete regions on a biomolecule array is determined by the total numbers of discrete regions of target biomolecules immobilized on the surface of a solid substrate, and, in some embodiments, the density is at least about 25/cm², at least about 50/cm², at least about 100/cm², at least about 500/cm², or at least about 1,000/cm². In some embodiments, each of the biomolecule arrays of the system comprises at least about two different immobilized biomolecules, at least about four different immobilized biomolecules, at least about 10 different immobilized biomolecules, at least about 100 different immobilized biomolecules, at least about 1,000 different immobilized biomolecules, or at least about 10,000 different immobilized biomolecules. For instance, in some embodiments,the biomolecule arrays comprise up to about 20,000, up to about 30,000, or up to about 40,000 immobilized biomolecules. In some embodiments, the average spot size of the biomolecule spots on the array is about 1 mm² or less.

In some embodiments, the multi-array system is a double-array system comprising two biomolecule arrays, one immobilized on each of the first and second surfaces of the system. In alternative embodiments, the multi-array system comprises more than two biomolecule arrays. For instance, in some embodiments, the multi-array system comprises at least four biomolecule arrays.

A “biomolecule” is any molecule or complex of molecules of biological interest. The term “biomolecule” includes, but is not limited to, polynucleotides, polypeptides or peptides, cells, and ligands (for instance, carbohydrate ligands). The term “biomolecule” also includes other entities which are known to bind to or otherwise react with polynucleotides, polypeptides or peptides, or cells, or which are entities that are thought to potentially bind to or otherwise react with polynucleotides, polypeptides or peptides, or cells (for instance, small molecule drug candidates). In some embodiments, the biomolecules of the biomolecule array are polynucleotides. In some embodiments, each of the biomolecule arrays of the system comprises at least about 10 different polynucleotides, at least about 100 different polynucleotides, at least about 1,000 different polynucleotides, or at least about 10,000 different polynucleotides. For instance, in some embodiments, the polynucleotide arrays comprise up to about 20,000, up to about 30,000, or up to about 40,000 polynucleotides. In some embodiments, each of the immobilized polynucleotides corresponds to a portion of a gene (e.g., a human gene or a mouse gene). Alternatively, the biomolecules of the array may be immobilized proteins, such as, but not limited to, antibodies.

In some embodiments, the multi-array system is a double-array system comprising two microarrays, each of which comprises 20,000-25,000 different oligonucleotides corresponding to portions of human gene sequences. In some embodiments, this double-array system is used to survey the entire human genome simultaneously in a single hybridization reaction.

Accordingly, in some embodiments, the biomolecule arrays are polynucleotide arrays. In some other embodiments, the biomolecule arrays are protein arrays. In some embodiments, the protein arrays are antibody arrays.

The terms “polynucleotide” and “oligonucleotide” are used interchangeably herein to refer to a polymeric form of nucleotides of any length and any origin, either ribonucleotides or deoxyribonucleotides. This term refers only to the primary structure of the molecule. Thus, this term includes double- and single-stranded DNA and RNA, as well as DNA-RNA duplexes. It also includes known types of modifications, for example, labels which are known in the art, methylation, “caps”, substitution of one or more of the naturally occurring nucleotides with an analog, internucleotide modifications such as, for example, those with uncharged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention (e.g., disulfide bond formation, glycosylation, lipidation, acetylation, phosphorylation, or any other manipulation or modification, such as conjugation with a labeling component). Also included within the definition are, for example, polypeptides containing one or more analogs of an amino acid (including, for example, unnatural amino acids, etc.), as well as other modifications known in the art.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, and any other modified configuration of the immunoglobulin molecule that comprises an antigen recognition site of the required specificity.

In some embodiments, the first biomolecule array of the multi-array system comprises a set of biomolecules that is different from the biomolecules that the second biomolecule array comprises. For instance, in some embodiments, at least one biomolecule on the first biomolecule array is different from the second biomolecule array. For instance, in some embodiments, the first biomolecule array comprises a first set of about 20,000 different polynucleotide sequences derived from the human genome, whereas the second biomolecule array comprises a different set of about 20,000 different polynucleotide sequences derived from the human genome. In some embodiments, every biomolecule of the first biomolecule array is different from every biomolecule of the second array. In some embodiments, the first biomolecule array and the second biomolecule array of the multi-array system are substantially identical. In some embodiments, the first biomolecule array and the second biomolecule array of the multi-array system are identical.

Methods and materials for derivatization of solid substrates for the purpose of immobilizing oligonucleotides are known to those skilled in the art. Oligonucleotides may be affixed, immobilized, provided, and/or applied to the surface of the solid substrate using any available means to fix, immobilize, provide and/or apply oligonucleotides at a particular location on the solid substrate. The various species may be placed at specific sites using ink jet printing (U.S. Pat. No. 4,877,745), photolithography (see, U.S. Pat. Nos. 5,919,523, 5,837,832, 5,831,070, 5,770,722 and 5,593,839), silk printing, offset printing, stamping, mechanical application with micropipets using an x-y stage or other rastering technique, or any other method which provides for the desired degree of accuracy and spatial separation in placing the bound component. Oligonucleotides may also be applied to a solid support as described in Brown and Shalon, U.S. Pat. No. 5,807,522. Additionally, oligonucleotides may be applied to a solid substrate using a robotic system, such as one manufactured by Affymetrix (Santa Clara, Calif.), Genomic Solutions (Ann Arbor, Mich.), Genetix, Inc. (Boston, Mass.), Labman Automation (North Yorkshire, UK), and Radius Biosciences, Inc. (Medfield, Mass.). For instance, in some embodiments, the biomolecule arrays are printed using the Affymetrix 427 Arrayer from Affymetrix, the QArrayMax, QArray2, or QArrayMin microarrayer from Genetix, Inc., the OmniGrid Micro, OmniGrid Accent, MicroGrid Compact Plus, MicroGrid, OmniGrid 100, or OmniGrid 300 microarrayer from Genomic Solutions, or the Radius 3XVP Arrayer from Radius Biosciences.

In some embodiments, when constructing the polynucleotide array it is desirable to draw a plurality of samples simultaneously, for example, from a 96 well plate. For this purpose, an array of tips can be mechanically assembled, where the tips may provide for a vacuum, be magnetic or other means for gripping and transferring a particle. The device simultaneously introduces the tips into the plurality of wells and each tip withdraws a particle. The device is then reoriented, if necessary, to mirror the orientation of the biomolecule arrays, and moved into juxtaposition with the biomolecule arrays on the slide. The particles are then released into defined regions on the solid substrate, where orientation and release is monitored with a laser beam or video camera.

In some embodiments, a microcomputer-controlled plotter delivers the oligonucleotides to the predetermined regions of the solid substrate. The sequences of oligonucleotides are selected through the application of a computer algorithm capable of using data describing gene sequences. The oligonucleotides are prepared using standard methods and presented in an array whereby the address of each of the oligonucleotides in the array is known. The information for the oligonucleotide sequences, the gene from which it is derived, and the position on the slide to which the oligonucleotide is applied being stored in the memory of a computer file generated via the computer algorithm.

The plotter, also known as a microarraying apparatus or microarrayer, removes a predetermined aliquot of solution containing the prepared oligonucleotides from their respective position in the presentation format provided by the manufacturer (or preparer). On a smooth impermeable surface, such as glass, it is possible to achieve a resolution of about 100 microns, for example, by using an arrayer employing a ring-and-pin mechanism for delivery of discrete and reproducible volumes of material to predetermined positions in the array.

The biomolecule arrays may be immobilized on the solid substrates through any of the many variety of methods known to those of ordinary skill in the art. The biomolecules of the array may be directly immobilized on the solid substrate surface or may be indirectly immobilized on the solid substrate surface, using one or more intermediates, which serve as bridges between the bound component and the solid substrate. The biomolecules of the arrays may be either covalently immobilized or non-covalently immobilized on the surface of the substrates. In general, where a molecule is to be covalently bonded to the solid substrate surface, the surface may be activated using a variety of reactive functionalities, depending on the nature of the bound component and the nature of the surface of the solid substrate.

For example, one may use a variety of approaches to bind an oligonucleotide to a solid substrate surface. By using chemically reactive solid substrates, one may provide for a chemically reactive group to be present on the nucleic acid molecule, which will react with the chemically active solid substrate surface. One may form silicon esters for covalent bonding of the nucleic acid to the surface. Instead of silicon functionalities, one may use organic addition polymers, e.g. styrene, acrylates and methacrylates, vinyl ethers and esters, and the like, where functionalities are present which can react with a functionality present on the nucleic acid. Amino groups, activated halides, carboxyl groups, mercaptan groups, epoxides, and the like, may also be provided in accordance with conventional ways. The linkages may be amides, amidines, amines, esters, ethers, thioethers, dithioethers, and the like. Methods for forming these covalent linkages may be found in U.S. Pat. No. 5,565,324 and references cited therein.

Methods for forming protein arrays are also known to those in the art. For instance, techniques for synthesizing peptides on solid substrates has been reported in the art. For instance, see U.S. Pat. No. 5,143,854 and U.S. Pat. No. 6,506,558. Methods for immobilizing proteins on solid substrate surfaces has also been described in U.S. Pat. No. 6,475,809, U.S. Pat. No. 6,475,808, U.S. Pat. No. 6,406,921, U.S. Pat. No. 6,329,209, and U.S. Pat. No. 6,365,418.

The multi-array system of the present invention comprises a spacer. The spacer separates the first and second solid substrates and fixably positions the first and second solid substrates so that a surface of the first solid substrate faces a surface of the second solid substrate and so that there is a reaction chamber comprising a fluid receiving space between the first and second solid substrates. In some embodiments, the first and second substrates are positioned so that the first and second biomolecule arrays also face each other across the fluid-receiving space of the reaction chamber.

In some embodiments, the spacer is a single unit. Alternatively, the spacer comprises multiple subunits. In some embodiments, the subunits of a multiple subunit spacer are in contact with each other in the multi-array system. In other embodiments, the units are positioned at discrete locations within the multi-array system. In some embodiments, the spacer has been injection molded, extruded or machined to the desired shape.

The spacer may be comprised of any material suitable for fixably positioning the first and second solid substrates according to the invention. For instance, in some embodiments, the spacer is a rigid material. In an alternative embodiment, the spacer is instead comprised of a semi-rigid material. In some embodiments, the spacer is made of plastic or rubber. In some embodiments, the spacer is made of Teflon®. In some embodiments, the spacer is water repellant (i.e., water resistant). In alternative embodiments, the spacer is water impermeable. In some embodiments, the material from which the spacer is made is hydrophobic.

In some embodiments the spacer is a holder which holds both the first and second solid substrates in position. In some embodiments, the holder comprises a first and second groove. In some embodiments, the multi-array system comprises a first solid substrate having a first edge and a second solid substrate having a second edge, wherein the spacer holds the first edge in its first groove and the second edge in its second groove. In some embodiments, the holder is made of precision-machined hard plastic. In some embodiments, the holder is fabricated from such polymeric materials as polypropylene, polystyrene, polycarbonate, polysulphone, Teflon®, or the like.

In some alternative embodiments, the spacer contacts the first and second solid substrates only on the opposing solid substrate surfaces (the first and second surfaces) of the multi-array system.

In some embodiments, the spacer is removably adhered to the first and/or second surfaces. In some embodiments, the spacer comprises a water-repellant material that is removably adhered to the first and/or second surfaces.

In some embodiments, the spacer is made of a rubber. In some embodiments, the rubber is hard, but flexible. In some embodiments, the rubber is a soft rubber. In some embodiments, the rubber is temperature resistant and/or is autoclavable. In some embodiments, the spacer is made of a silicone rubber. In some embodiments, the rubber is water-repellant (i.e., water resistant). In some embodiments, the rubber is water impermeable. In some embodiments, the rubber is hydrophobic. In some embodiments, the rubber is not hydrophobic. In some embodiments, the rubber is a water impermeable soft rubber which remains water impermeable following the insertion (and subsequent removal) of a thin syringe needle (such as a 27 gauge needle or a 28 ½ gauge needle) through the rubber.

In some embodiments, the spacer is made from a gasket material, such as rubber gasket material and/or silicone sealing gasket material or like material known in the art. In some embodiments, the spacer is a gasket made from a rubber-like plastic. The gasket material may or may not be compressible, but in some embodiments, a slightly compressible gasket forms a better seal. However, in some embodiments, the gasket material is not so compressible that in its final assembly the slides are touching or separated by less than about a micron. In some embodiments, the gasket material is not so compressible that in its final assembly the slides are touching or separated by less than about 0.1 mm. Also, in some embodiments, any compression that might occur is controlled so that the volumes will not vary widely from one device to another of the same make. In some embodiments, the spacer is made of gasket material that is impermeable to water. In some embodiments, the gasket material is hydrophobic. In other embodiments, the gasket material from which the spacer is made is not hydrophobic. A variety of suitable rubber gasket materials are available in the art for use as spacers in the present invention.

In some embodiments, the spacer is a U-shaped gasket. In some embodiments, the spacer has a closed periphery creating an internal cavity. In other embodiments, the spacer is an O-shaped gasket. In some embodiments, the gasket is circular, rectangular, or square. In some embodiments, the spacer is a gasket (e.g., a rubber gasket), and the reaction chamber is enclosed by the first surface of the first solid substrate, the second surface of the second solid substrate, and the spacer.

In some embodiments, the spacer is a silicone gasket. In some embodiments, the spacer is a silicone gasket and the reaction chamber is enclosed by the first and second surfaces and the spacer. In some embodiments, the spacer is a silicone gasket and the reaction chamber is enclosed by the first and second surfaces and the spacer, except for two openings, each provided by a syringe needle that penetrates an exposed edge of silicone gasket and terminates in the fluid-receiving space of the reaction chamber.

In some embodiments, the spacer is a commercially available gasket that has been adapted for use in the present invention. For instance, in some embodiments, the spacer is the commercially available large EasiSeal™ chamber from Hybaid (Catalogue #HB-OS-SSEZ3E, Thermo Electron Corporation, Waltham, Mass.) or another EasiSeal™ chamber. In some embodiments, the gasket is used as in the rectangular form in which it is supplied. In other embodiments, one side of the rectangular gasket is cut off to make a U-shaped gasket. Prior to use, the “backing” of the gasket is removed to expose the adhesive. The U-shaped gasket is then positioned on one substrate, such as a glass slide. The backing is then removed from the other side of the gasket (exposing its adhesive). A second glass slide is then subsequently laid on it. The sandwich of plates and gaskets is then squeezed tight. In some embodiments, the sandwich is held in place with shims. In some alternative embodiments, the gasket used as a spacer in the multi-array system is another EasiSeal™ chamber from Hybaid or a gasket equivalent to an EasiSeal™ chamber that is obtained from another source.

In some embodiments, the spacer used in the multi-array system is a commercially available gasket from the SecureSeal™ Hybridization Chamber (e.g., Cat. #s SA200, SA500, and SA4545), SecureSeal™ Imaging Chamber (also known as SecureSeal™ Spacers; e.g., Cat. #SS1×20), CoverWell™ incubation chamber (e.g., Cat. #s PC220 and PC500), or CoverWell™ imaging chamber (e.g., Cat. #s PCI-1.0 and PCI-A-2.0) products offered by Grace Bio-Labs (Grace Bio-Labs, Inc., Bend, Oreg.), or the equivalent of such a gasket that is obtained from another source. In some embodiments, these gaskets are soft rubber gaskets, such as soft silicone rubber gaskets. In some embodiments, the gaskets are made from a rubber-like plastic. Generally, the gaskets can be removably adhered to substrates such as two glass slides to form stable, water-tight seals around an interior reaction chamber. The gaskets are temperature resistant and are autoclavable. In some embodiments, the gaskets form the seal with a substrate such as glass in the absence of an adhesive or sealant. In other embodiments, an adhesive is present on the gasket (such as, for instance on Cat. #SA500). In some cases, prior to use, the “backing” of the gasket is removed to expose the adhesive. In still other embodiments, an adhesive or sealant is added to the commercially available gaskets prior to use in the present invention. The gaskets typically range from about 0.1 mm to about 3 mm in thickness. One example of a commercially available, rectangular gaskets has exterior dimensions of about 44 mm by about 25 mm and interior dimensions of about 40 mm by about 20 mm.

As will be evident to one of ordinary skill in the art, the desired thickness of the spacer will depend on the nature of the material used for the spacer as well as the dimensions of the solid substrates and the bimolecule arrays. In some embodiments, the spacer is from about 0.001 mm to about 10 mm thick. In other embodiments, the spacer is from about 0.01 mm to about 5 mm thick. In further embodiments, the spacer is from about 0.1 mm to about 3 mm thick. Accordingly, in some embodiments, the spacer is a gasket that is from about 0.1 mm to about 3 mm thick.

In some embodiments, an inert, reversible adhesive is used to affix the spacer to the opposing solid substrate surfaces of the system. In some embodiments, the inert, reversible adhesive is an acrylic adhesive (optionally, a biocompatible acrylic adhesive). In some embodiments, the inert, reversible adhesive is a silicone sealant (optionally, a medical grade silicone sealant). The adhesive is preferably not so strong as to make the disassembly of the multi-array system difficult or to risk damage to the substrates or biomolecule arrays. For instance, in some embodiments, a silicone sealant is used to removably adhere a silicone gasket to both the first and second substrate.

In some embodiments, the spacer used in the multi-array system will form a watertight seal with both the first and second surfaces of the solid substrates. In alternative embodiments, an adhesive used to adhere the spacer to the first and second solid substrates will create a watertight seal at the interface between the spacer and the solid substrates. Thus, in some embodiments, the adhesive of the system acts as a sealant. In still other embodiments, the multi-array system further comprises a sealant that doesn't act as an adhesive, but which nonetheless functions to form a watertight seal between the spacer and the first solid substrate and/or between the spacer and the second solid substrate. In still other embodiments, the multi-array system further comprises a water-impenetrable liner in the reaction chamber that forms a watertight seal between the spacer and the first solid substrate. In some embodiments the liner is made from Teflon® or a similar substance.

The materials used for the spacer and any adhesives, sealants, or liners used therewith should be sufficiently inert that they do not impart substances to the surrounding area that interfere with hybridization and other reactions desired to take place in the chamber. In some embodiments, the spacer(s) and any adhesives, sealants, or liners used therewith are medical grade. The materials used for the spacers, adhesive, sealants, or liners of the invention should be stable through the full range of temperatures contemplated for the assay for which the multi-array system is designed. For gene expression work (e.g., polynucleotide hybridization assays), for instance, the temperatures used in the assays could range in some embodiments from about room temperature to 60° C. In some other embodiments where the assay involves PCR, the temperature range could be from about 4° C. to about 99° C., and the materials used in the system components do not greatly expand, contract, or otherwise lose functional properties at temperatures between about 0°C. and about 100° C.

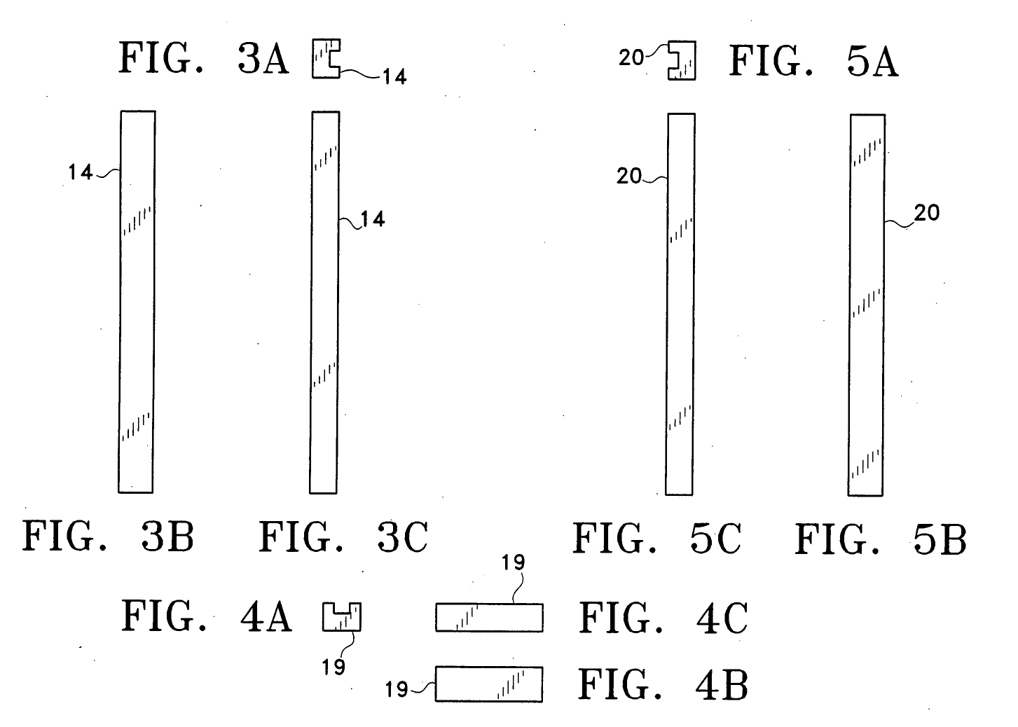

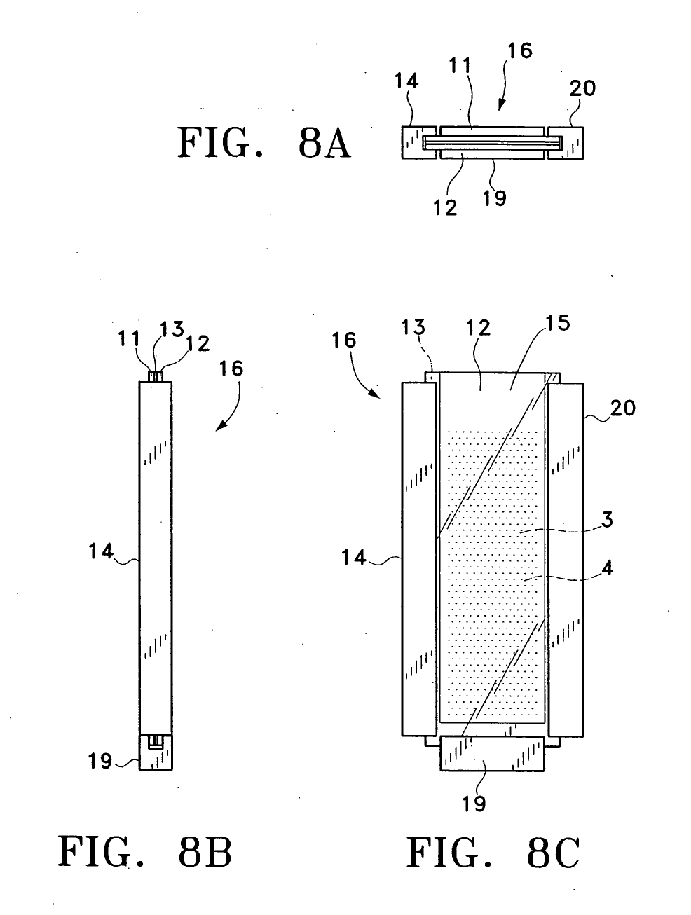

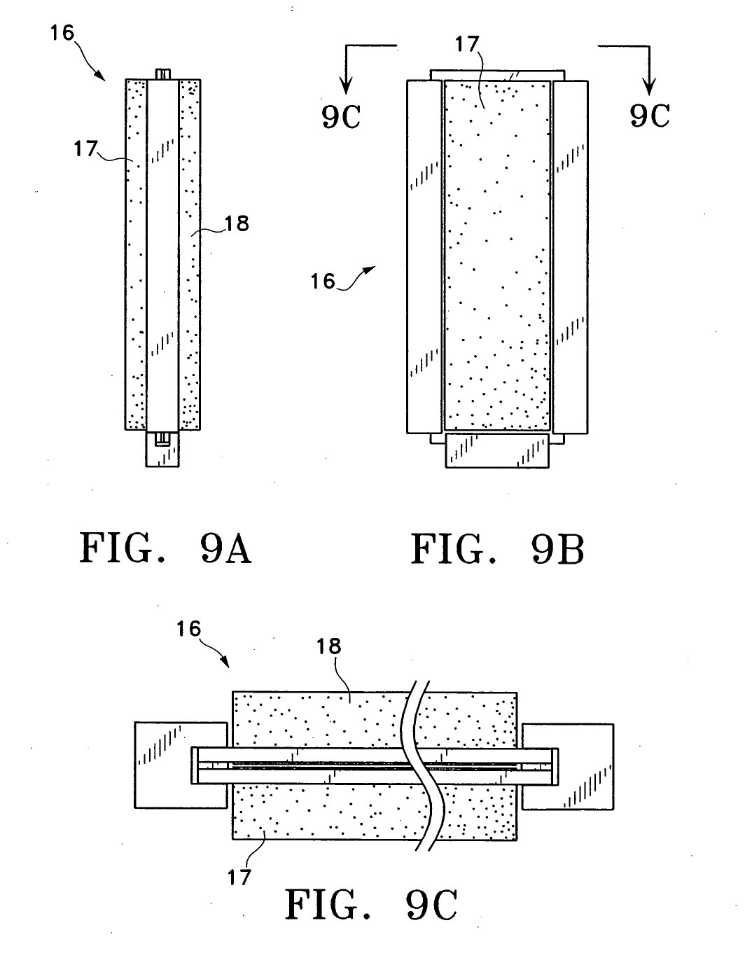

In some embodiments, the multiple-array systems of the present invention further comprise one or more devices which act to help maintain the overall structure of the assembled system. In addition to lending stability to the assembled system, in some embodiments these devices also often help increase the watertightness of the system by compressing the components together and/or compressing individual components (such as a gasket). In some embodiments, the devices contact the outer surfaces of each of the substrates. These devices may be shims. A non-limiting example of such shims are those pictured in FIGS. 3, 4, and 5 (and shown in place in a double-array system in FIGS. 8 and 9). In some embodiments, the shims are fabricated out of plastic, metal, hard rubber or like materials. In some embodiments, the shims partially encase the edges of the substrate, holding the substrates within a certain distance from each other. In some alternative embodiments, the devices are clamps, such as those made from metal or plastic. Still further embodiments will be readily apparent to one of ordinary skill in the art.

In some embodiments, the first and second surfaces are each segmented into at least two defined regions, each of the at least two defined regions comprising a biomolecule array, wherein the spacer surrounds each defined region and creates a watertight seal, the spacer being removably adhered to the first and second surfaces. Accordingly, each region serves as a reaction chamber. Examples of slides with removable chambers are known to those in the arts of biomedical research and pathology. In some embodiments, the defined regions encompassing the biomolecule arrays are from about 0.5 mm to about 20 mm, from about 0.5 mm to about 10 mm in each dimension, or from about 2.0 to about 8.0 mm in each dimension. There may be as few as two regions per apparatus or slide and as many as 8, 16, 32, 64, 128, 256 or more regions per slide. In some embodiments, each defined region comprises up to several hundred different biomolecules in an array.

In some embodiments, the reaction chamber of the multi-array system is substantially enclosed by the first and second surfaces of the solid substrates and by the spacer. For instance, in some embodiments, the reaction chamber is at least about 50%, at least about 55%, at least about 60%, at least about 65%, at least about 70%, at least about 75%, at least about 80%, at least about 85%, at least about 90%, at least about 95% enclosed, or at least about 98% enclosed. In an alternative embodiment, the reaction chamber is totally enclosed (i.e., about 100% enclosed) by the first and second surfaces of the solid substrates and the spacer.

In some embodiments, the reaction chamber is walled on two opposing sides by the solid substrates and on three of the remaining four sides by the spacer or spacers. In some other embodiments, the reaction chamber is walled on two opposing sides by the solid substrates and on four of the remaining four sides by the spacer or spacers.

In some embodiments, the multi-array system comprises a reaction chamber having one or more openings. In some embodiments, the one or more openings are sealable. In some embodiments, the multi-array system comprises a reaction chamber with one opening. In some other embodiments, the multi-array system comprises a reaction chamber with two or more openings, three or more openings, or four or more openings. In some embodiments, the one or more openings are temporary openings into an otherwise enclosed reaction chamber.

In some embodiments, the reaction chamber comprises both a distal and a proximal end and comprises an opening at its proximal end. In some embodiments, the opening is sealable. In some embodiments, the reaction chamber comprises a second opening at the distal end. In some embodiments, the second opening is also sealable.

In some embodiments, the opening of the reaction chamber is sealed (or resealed) by any of a number of ways known to those of ordinary skill in the art. A solid lid or seal, such as one fabricated from material identical to that used for the spacer (or different form that used for the spacer) is one option. Another option is a liquid seal. For instance, the opening in the reaction chamber may be sealed for purposes of the invention by coating the liquid in the reaction chamber with a layer of mineral oil. In some embodiments, this layer of mineral oil is preferred to that of a solid lid because it allows for expansion or contraction of the reaction mix volume when heated or cooled and provides easy access to the solution if the user needs to add or remove materials while the reaction is running.

In some embodiments, an opening in an otherwise enclosed reaction chamber is generated by insertion of a needle from outside of the multi-array system into the reaction chamber. In some embodiments, a second opening into the reaction chamber is generated by insertion of a needle from outside the multi-array system into the reaction chamber. In some embodiments, the needle(s) is inserted into the reaction chamber through a rubber spacer, optionally a rubber spacer which reseals after removal of the needle(s). In some embodiments, where two needles are inserted, one of the needles serves the purpose of a vent, whereas the other needle is used for insert of a fluid into the fluid receiving spacer of the reaction chamber. In other embodiments, the two needles are used as part of a continuous flow system whereby fluid enters the fluid-receiving space of the reaction chamber through one of the needles and is removed through the other needle (useful, for instance, in real time assay analysis, etc.). In some embodiments, the insertion of the needles is temporary, and when the needles are removed, the openings either reseal automatically (for instance, because of the nature of the rubber from which the spacer is made) or are resealed. One of ordinary skill in the art will be readily able to choose a gauge of needle that is appropriate for a particular multi-array system. For instance, for use with a multi-array system comprising a rectangular silicone gasket that is approximately 1 mm thick sandwiched between two glass slides that are also about 1 mm thick, suitable needles include, but are not limited to, a 27 gauge needle or a 28 ½ gauge needle.

In some embodiments, the average distance in the reaction chamber between the first surface on the first solid substrate and the opposing, second surface on the second solid substrate is between about 0.01 mm and about 5 cm. In some other embodiments, the average distance is between about 0.01 mm and about 2 cm. In some other embodiments, the average distance in the reaction chamber between the first surface on the first solid substrate and the opposing, second surface on the second solid substrate is between about 0.001 mm and about 10 mm. In some embodiments, the average distance is between about 0.01 mm and about 5 mm. In some embodiments, the average distance is between about 0.01 mm and about 3 mm. In some embodiments, the average distance is between about 0.01 mm and about 2 mm. In some embodiments, the average distance is between about 0.03 mm and about 2 mm. In some embodiments, the average distance is between about 0.05 mm and about 1 mm. In yet another embodiment, the average distance is between about 0.05 mm and 0.5 mm. In still another embodiment, the average distance is between about 0.1 mm and about 0.5 mm. In some embodiments the distance is less than about 5 mm. In some embodiments the distance is greater than about 0.01 mm. In some embodiments, the average distance between the first surface on the first solid substrate and the opposing, second surface on the second solid substrate is between about 0.1 mm and about 3 mm.

In some embodiments, the volume of the reaction chamber is between about 5 μl and about 10 ml. In some embodiments, the volume is between about 20 μl and about 5 ml. In some embodiments, the volume is between about 50 μl and about 4 ml. In some embodiments, the volume is between about 100 μl and about 4 ml. In some embodiments, the volume is between about 100 μl and about 3 ml. In still another embodiment, the volume is between about 50 μl and about 1 ml. In some embodiments, the volume of the reaction chamber is less than about 20 ml, less than about 10 ml, less than about 5 ml, less than about 4 ml, less than about 3 ml, less than about 1 ml, less than about 500 μl, or less than about 250 μl.

For instance, if a gasket spacer derived from the large Hybaid chamber is prepared and used in the multi-array system (see above), then the reaction chamber is 19 mm wide and 60 mm deep and the volume of the reaction chamber is less than 1 ml (estimates range from about 125 μl to over 500 μl). As another non-limiting example, the reaction chamber of a multi-array system comprising a rectangular-shaped gasket spacer from Grace Bio-Labs having internal dimensions of about 40 mm by about 22 mm by about 0.7 to 1.0 mm and two glass slides as substrates also has an internal fluid-receiving volume of less than one 1 ml (estimates range from about 150 μl to about 620 μl).

In some embodiments, the multi-array system further comprises fluid in the reaction chamber. For instance, in some embodiments, reaction chamber contains a buffered aqueous solution. In some embodiments, the fluid is a reaction mixture. In some embodiments, the fluid in the reaction chamber comprises polynucleotides, nucleotides, and/or a protein. In some embodiments, the fluid is an assay solution (a solution useful in performing a desired assay with the multi-array system). In some embodiments, an assay solution comprises biomolecules which will be assayed for binding or some other form of reaction with the arrayed biomolecules of the first and second biomolecule arrays. Alternatively, the assay solution consists essentially of buffers and/or salt solutions appropriate for use with the multi-array system and to which biomolecules can be added by those using the system. In some embodiments, the multi-array system further comprises a fluid in the reaction chamber, wherein the fluid is an assay solution selected from the group consisting of a hybridization solution or a polymerase chain reaction solution mixture. If the multi-array system is to be used to perform hybridization reactions, in some embodiments, the assay fluid is a hybridization solution (i.e., a solution useful for performing a hybridization assay). In some embodiments, the hybridization solution contains polynucleotide molecules (e.g., DNA or RNA probes) to be assayed for hybridization to the first and second biomolecule arrays as well as suitable salts, buffers, and the like for promoting the desired hybridization of polynucleotides to complementary sequences. In some alternative embodiments, DNA amplification on the biomolecule arrays is desired, and the assay solution is a polymerase chain reaction (PCR) solution mixture comprising DNA polymerase, nucleotides, oligonucleotide primers, and/or appropriate salt and buffer levels. In still another embodiment, the assay solution comprises prospective ligands, such as proteins, to be delivered to antibodies on the first and second biomolecule arrays. Accordingly, in some embodiments, the assay solution will be understood to contain salts and buffers that support binding of the prospective ligands to the antibodies. Such assay solutions are well-known to those of ordinary skill in the art.

In some embodiments, the volumes of fluid which are contained in the reaction chamber of the multi-array systems described herein may be slightly smaller than the volumes of the reaction chambers. For instance, in some embodiments a layer of mineral oil of about a few millimeters in depth is used on top of the fluid in the reaction chamber. Thus, in some embodiments, the use of mineral oil may decrease the volume of the fluid by approximately one eighth. In some alternative embodiments the entire volume, or nearly the entire volume, of the reaction chamber is filled with fluid.

The invention also provides kits (also referred to as “articles of manufacture”) which comprise the multi-arrays systems described herein or components of the multi-array system and/or which are useful in the methods described herein. In some embodiments, the kit comprises a multi-array system described herein. In some embodiments, the multi-array system is pre-assembled in the kit. In some embodiments, the multi-array system is not pre-assembled in the kit. In some embodiments, the kit comprises only select components of the multi-array system described herein.

In some embodiments, the kit not only comprises a multi-array system described herein, but also comprises one or more reagents. In some embodiments, the kit comprises a multi-array system described herein (either preassembled or not preassembled) and a solution for use in performing an assay with the multi-array system. In some embodiments, the invention provides a kit for use in performing an assay where the kit comprises a multi-array system described herein and an assay solution. The multi-array system may or may not be pre-assembled in the kit. In some embodiments, the invention provides a kit comprising the spacer described herein and an assay solution. In some embodiments, the kit comprises the spacer described herein and instructions for assembling and using a multi-array system in accordance with the methods of the invention. The solid substrates bearing the biomolecule arrays may or may not be included in such a kit.

In some embodiments, the kit comprises a multi-array system described herein and suitable packaging and/or instructions for use of the multi-array system in any of the methods described herein. In some embodiments, the kit comprises a multi-array system described herein and instructions regarding the use of the multi-array system in at least one step of an assay. In some embodiments, the instructions are on the packaging of the kit. In other embodiments, the instructions are on an insert contained within the kit.

In some embodiments, regulation of the temperature of the reaction chambers within the multi-array systems described herein is desirable. Although the multi-array system described herein does not necessarily, need to be used in conjunction with a thermal cycler, a thermal cycler is, in some embodiments, useful for controlling the temperature of the solid substrate surfaces on which the biomolecule arrays are immobilized and/or the reaction chamber containing the biomolecule arrays. For instance, in a hybridization assay or other binding assay, maintaining even temperature control at the site of the biomolecule arrays may be desirable and can be effected with a thermal cycler. If the reaction carried out at the site of the biomolecule arrays involves PCR amplification, then the use of a thermal cycler in conjunction with the system described herein is also desirable. In some alternative embodiments of the invention, temperature regulation of the multi-array system is maintained by a water bath or air system, rather than a thermal cycler.

Thus, in one aspect, the invention provides an apparatus comprising a multi-array system described herein and a temperature control unit, wherein the temperature control functions to alter the temperature of the reaction chamber of the multi-array system. In some embodiments, the temperature control unit comprises instrumentation that allows the temperature of the reaction chamber to be altered in a controllable manner. In some embodiments, the temperature control unit is a thermal cycler. In some other embodiments, the temperature control unit is a water bath. In other embodiments, the temperature control unit is an air system, such as an air oven.

In some embodiments, the apparatus comprises the multi-array system described herein and a thermal cycler comprising a first temperature block, where the first temperature block is in thermal contact with the first solid substrate of the multi-array system. In some embodiments, the thermal cycler further comprises a second temperature block and the second temperature block is in thermal contact with the second solid substrate of the multi-array system.

In some embodiments, the temperature block, also known as a thermal block, and which may be a heating block, is positioned in direct contact with the first and/or second solid substrates of the multi-array system. The temperature block should be positioned so that it is in thermal contact with the solid substrate. In some embodiments, a temperature block is positioned on the opposite side of the solid substrate from the surface of the solid substrate bearing the biomolecule array.

Alternatively, a material which conducts heat is placed between a temperature block and the substrates of the multi-array system. For instance, in some embodiments, a brass plate is inserted between a substrate of the multi-array system and the temperature block.

Suitable thermal cyclers are known to those in the art which are or adaptable for use in the present invention. Depending on the configuration of the thermal cycler used and the position of any opening in the walls of the reaction chamber and the ability to seal such an opening, the multi-array system may be positioned in the thermal cycler in either a horizontal or vertical position (or any position in between). In some embodiments, a commercially available thermal cycler is used in which the thermal blocks portions of the cycler are typically used in the horizontal position, but for purposes of use with the multi-array system of the present invention, the thermal blocks are repositioned in a vertical position. For instance, the thermal block portion of the GeneAmp in situ PCR system 1000 (Applied Biosystems, Foster City, Calif.) can be unhooked from its base, thereby opening the machine as if to service its interior (in the manner designed). This positions the thermal block in a nearly vertical position compatible with use of the multi-array systems of the present invention.

In some embodiments, when using the multi-array system in a vertical or substantially vertical position with the opening to the top, mineral oil or a like substance is used to cover the exposed top surface of the fluid in the reaction chamber. Such a covering is often convenient in that it allows for the easy addition of reaction components to the fluid in the reaction chamber.

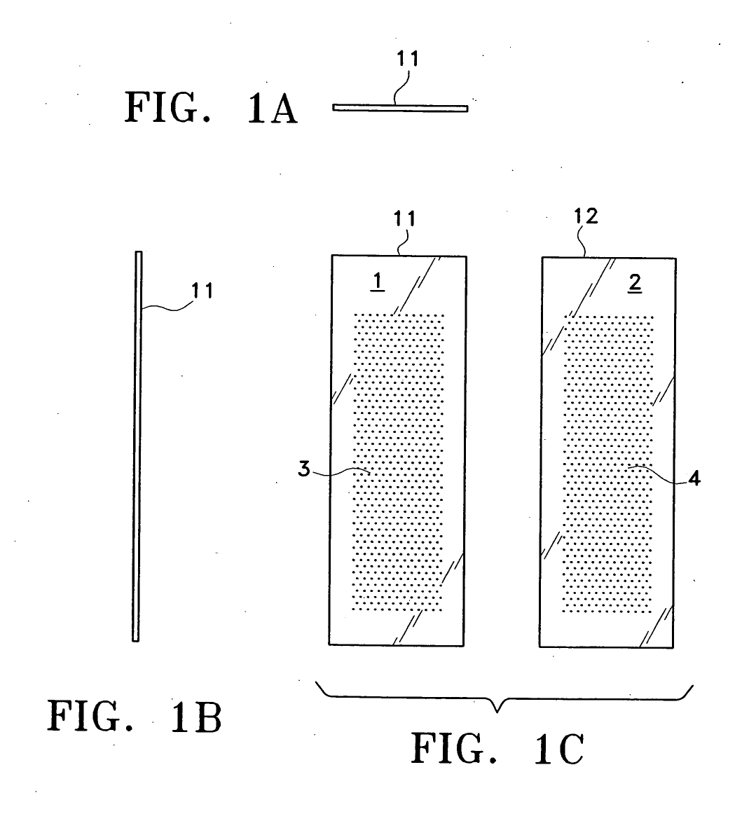

FIGS. 1-5 show various individual components of one embodiment of the subject invention, a double-array system formed by gaskets and held in place by shims (shown in FIGS. 8 and 9).

FIG. 1 shows top , side and front views of glass slides used in some embodiments of the invention. The top view of one glass slide 11 is shown in FIG. 1A, the side view of the glass slide 11 is shown in FIG. 1B, and the front views of two glass slides 11 and 12 are shown in FIG. 1C. In some embodiments of the invention, the glass slide 11 printed with biomolecule array 3 on a surface 1 is used in conjunction with a second glass slide 12 printed with another biomolecule array 4 on a surface 2. (Biomolecules arrays are shown as stippled areas.) Each of the glass slides 11 and 12 are approximately 75 mm long by 25 mm wide by 1 mm thick. Each of the biomolecule arrays 3 and 4 comprises 20,000 different oligonucleotides that have been printed on the surface of the slides.

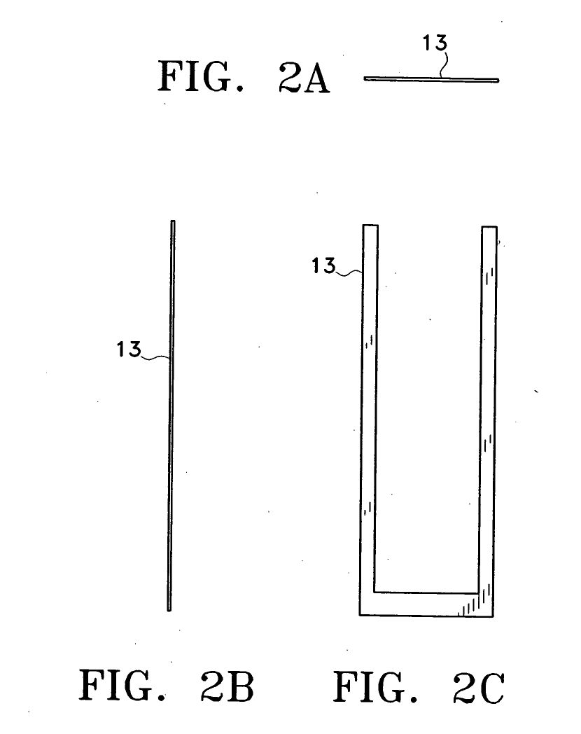

FIG. 2 shows top (2A), side (2B) and front (2C) views of a U-shaped gasket 13 which acts in the assembled invention to separate the slides 11 and 12 by a very small distance—here, 0.25 mm. Gasket 13 may or may not include an adhesive substance or other materials to aid in the construction and stability of the final assembly.

FIG. 3 shows top (3A), side (3B), and front (3C) views of a side shim 14 which is used in combination with bottom shim 19 (shown in FIG. 4) and side shim 20 (shown in FIG. 5) to maintain overall structure of the system.

FIG. 4 shows side (4A), bottom (4B), and front (4C) views of the bottom shim 19.

FIG. 5 shows top (5A), side (5B), and front (5C) views of the side shim 20.

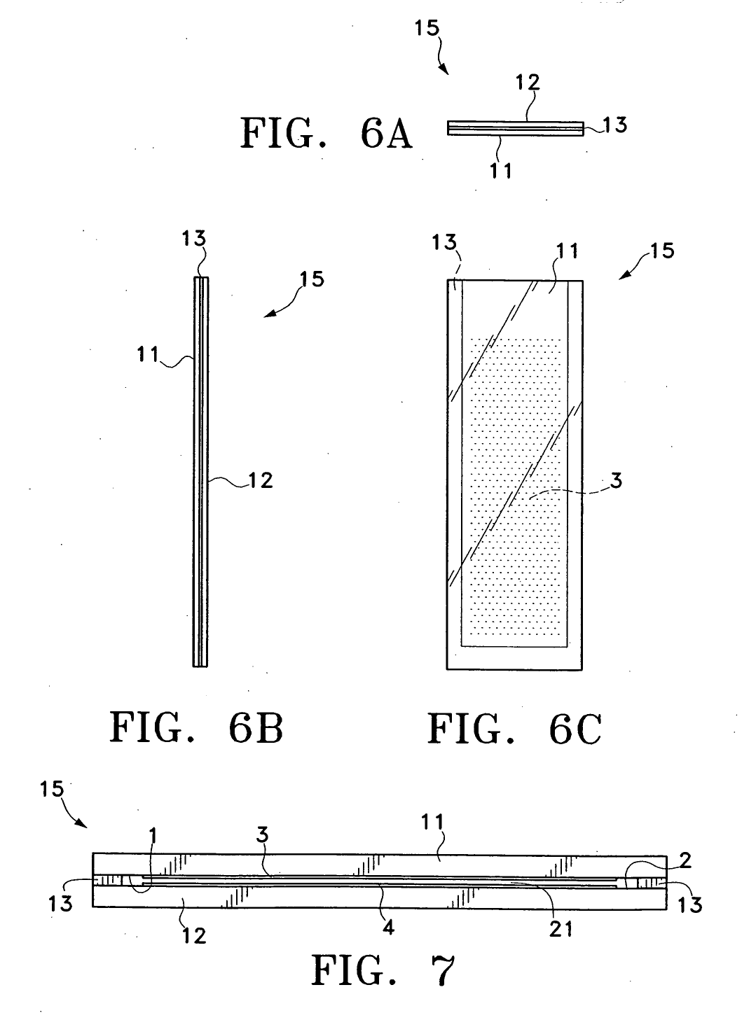

FIG. 6 through FIG. 9 illustrate, in part, the construction and use of a double-array system formed by gaskets and held in place by shims.

FIGS. 6 and 7 show one exemplary embodiment of the double-array system. FIG. 6 shows a top (6A), side (6B) and front view (6C) of a double-array system 15. FIG. 7 shows a close up of the top view of the double-array system 15 in greater detail. The double-array system 15 is formed by placing the gasket 13 along the three lower edges of one slide 11 bearing the biomolecule array 3. The other glass slide 12 bearing biomolecule array 4 is pressed against the gasket 13 so that the biomolecule array 4 on surface 2 of slide 12 faces biomolecule array 3 on surface 1 of slide 11. The inside dimensions and thickness of the gasket 13 will define the volume of the reaction chamber 21, shown in FIG. 7, although some compression of gasket 13 may cause the volume to be less than calculated. In the double-array system 15, the reaction chamber 21 is approximately 71 mm long by 21 mm wide by 0.25 mm thick creating a reaction chamber volume of 3.7 milliliters. (Some other embodiments (not shown) use gaskets as thin as about 10 microns, thereby producing a reaction chamber of approximately 150 microliters.) (Note that the biomolecule arrays 3 and 4 in FIGS. 6C and 7 are not shown to scale, but instead are enlarged for purposes of illustration.)

FIG. 8 provides an additional embodiment of the double-array system. In this embodiment, the double-array system comprises three shims 14, 19, and 20 of FIGS. 3, 4, and 5, respectively, in place around the assembled double-array system 15 shown in FIGS. 6 and 7. Top (8A), side (8B), and front (8C) views of the shim-fortified double-array system 16 are shown in FIG. 8. After assembly is complete, the reaction mix can be added to the double-array system 16. The volume of the reaction mix is such that biomolecule arrays 3 and 4 are completely submerged. Subsequent to addition of the reaction mix, a layer of mineral oil is added to cover the reaction mix to a depth of at least 1 mm and acts as a seal for the subsequent reaction(s).

FIG. 9 shows one embodiment of a fully assembled double-array system 16 of FIG. 8 in use. In the illustrated embodiment, temperature blocks 17 and 18 (for example, temperature blocks from Peltier Technology) are placed in direct contact with the back of each slide and used to heat the double-array system 16 from both slides. The temperature blocks 17 and 18 are operably connected to a thermal cycler or other temperature control unit (not shown). The side view of the apparatus is shown in FIG. 9A. The front view of the apparatus is shown in FIG. 9B. FIG. 9C shows a top elevational edge view taken along the line 9C-9C of FIG. 9B. (Note that the biomolecule arrays shown in FIG. 9C are not drawn to scale, but are instead enlarged for purposes of illustration.)

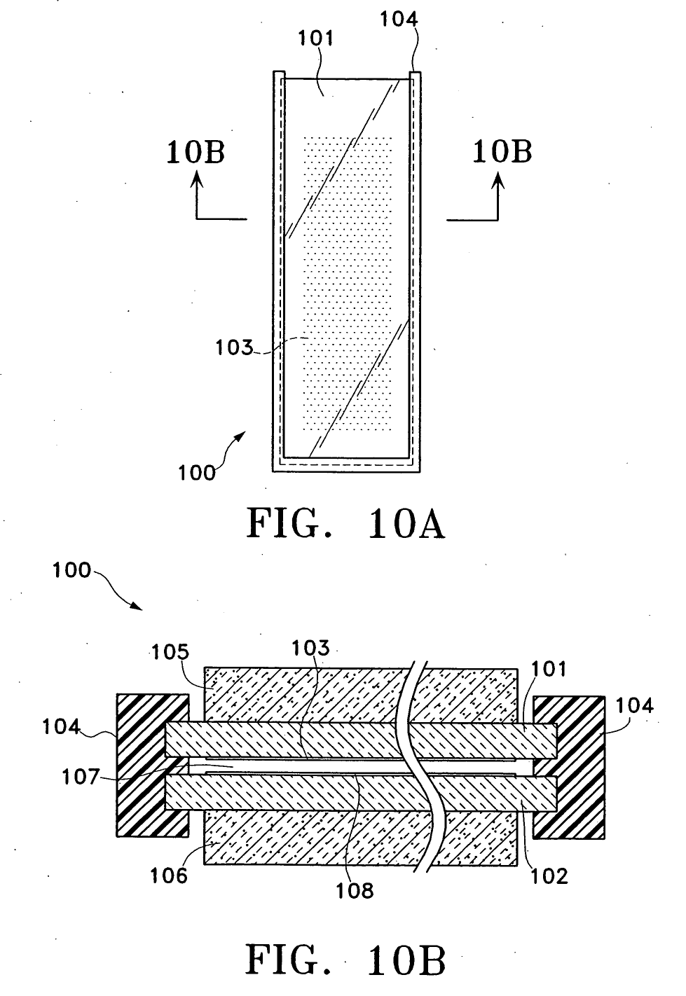

FIG. 10 illustrates a further specific embodiment of the subject invention. FIG. 10A shows the front view of a double-array system 100 in which two glass slides (including the glass slide 101 bearing the biomolecule array 103) are held in position by a plastic holder 104. FIG. 10B shows a cross-sectional view of the double-array system 100 taken along the line 10B-10B of FIG. 10A, although two temperature blocks 105 and 106 of a thermal cycler are additionally illustrated as sandwiching the double-array system. This cross-sectional view shows portions of the plastic holder 104, glass slide 101 (bearing biomolecule array 103) and glass slide 102 (bearing biomolecule array 108), temperature blocks 105 and 106, and a reaction chamber 107. (The remainder of the thermal cycler to which the temperature blocks 105 and 106 are operably connected is not shown.)

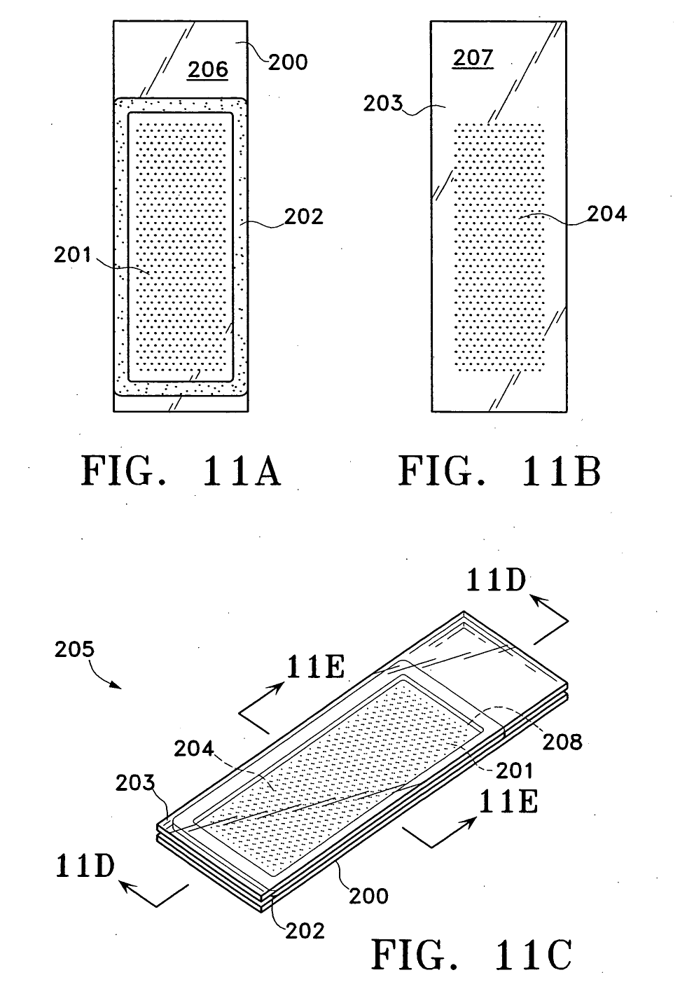

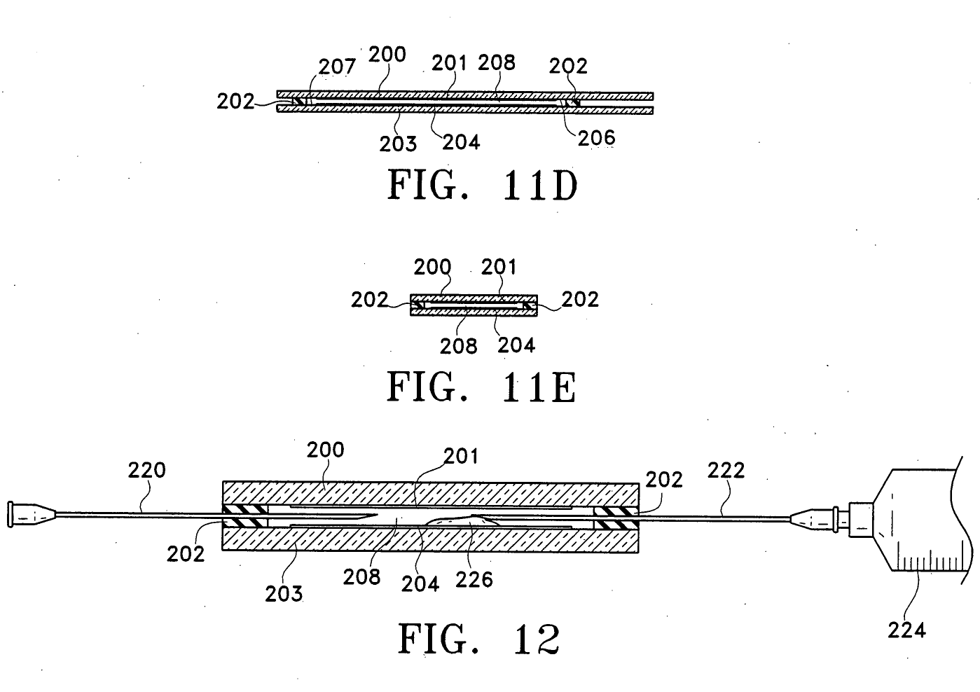

FIG. 11 shows different views or components of another embodiment of the invention, a double-array system 205 formed with a rubber, rectangular-shaped gasket 202 and two glass slides (25 mm×75 mm by about 1 mm) on which biomolecule arrays 201 and 204 have been printed. Each of the biomolecule arrays 201 and 204 contains 20,000 different immobilized oligonucleotide sequences. FIG. 11A shows the top view of a glass slide 200 on which a biomolecule array 201 has been printed on surface 206 and on which a rubber gasket 202 has been adhered. The rubber gasket is made from a soft rubber, is about 0.8-1.0 mm thick, and has exterior dimensions of about 44 mm by about 25 mm and interior dimensions of about 40 mm by about 20 mm. FIG. 11B shows the top view of a glass slide 203 on which a biomolecule array 204 has been printed on surface 207. FIG. 11C shows a perspective top view of the double-array system 205 which has been assembled from the components shown in FIGS. 11A and 11B by adhering the glass slide 203 of FIG. 11B to the rubber gasket 202 of FIG. 11A (an adhesive is present on the rubber gasket 202). This generates an enclosed reaction chamber 208 that has a volume estimated to be from about 150 μl to about 680 μl. FIG. 11D shows a cross-sectional view of the double-array system 205 taken along the line 11D-11D of FIG. 11C. The reaction chamber 208 can be seen in this figure. FIG. 11E shows a cross-sectional view of the double-array system 205 taken along the line 11E-11E of FIG. 11C. (Note that the biomolecule arrays shown in FIGS. 11D and 11E are not drawn to scale, but are instead enlarged for purposes of illustration.)

FIG. 12 shows a cross-sectional view of the double-array system 205 of FIG. 11C into which one syringe needle 220 has been inserted as a vent and a second syringe needle 222 attached to a syringe 224 has been inserted for transferring a fluid 226 (such as a hybridization solution) from the syringe 224 into the reaction chamber 208.

In other aspects, the invention provides methods of using the multi-array systems described herein in assays. These methods allow a single sample to be used to perform an assay on a plurality (i.e., two or more) of biomolecule arrays simultaneously. For instance, the invention provides a method of performing an assay on a plurality of biomolecule arrays simultaneously (and from the same sample volume), comprising using a multi-array system, apparatus, and/or kit described herein in the assay or at least one step of the assay. In some embodiments, the invention provides a method of performing an assay on two or more biomolecule arrays simultaneously, comprising using a multi-array system described herein in at least one step of the assay. In some embodiments the assay is a binding assay, such as a hybridization assay or an antibody-antigen assay. In some embodiments, the assay comprises a polymerase-mediated amplification reaction.

Methods of performing hybridization assays are well known to those of ordinary skill in the art and are easily adapted to use of the multi-array system. One example of the use of a multi-array system in an exemplary hybridization assay is provided below in Example 1. In some hybridization studies involving gene expression, RNA is first isolated from specific tissue samples. In some embodiments, this RNA is then subjected to reverse transcription using oligo-dT primers and fluorescently labeled dNTPs (sometimes Cy3 or Cy5 labeled) resulting in a DNA probe that is fluorescently labeled and has a complementary sequence to the original mRNA. In some alternative embodiments, RNA probes are used which have been produced by in vitro transcription of the DNA prepared from an RNA sample that has been reversed transcribed and then subjected to second-strand synthesis. A labeled nucleotide triphosphate (such as biotin-rCTP) is sometimes used in the in vitro transcription mix to produce labeled RNA probe.

In some embodiments, the next step of the hybridization assay is to hybridize the probe to the immobilized target DNA of two polynucleotide arrays. In some embodiments, this is done by first denaturing the probe with heat or a mild base to reduce secondary structures that may have formed and applying it onto the polynucleotide arrays by introducing the probe solution into the reaction chamber of the system like that shown in FIG. 8C or 11C. Suitable hybridization buffers for use in the hybridization of the probe to the arrays are well known to those of ordinary skill in the art. In some embodiments, the hybridization buffer is a high-stringency hybridization buffer solution such as the Buffer H provided in the HO5 ExpressChip™ DNA Microarray System kit provided by Mergen, San Leandro, Calif. as product #HO5-001, or its equivalent. A variety of hybridization buffers suitable for use with microarrays are known to those in the art and many versions are available commercially from a variety of sources (e.g., from Telechem International, Inc., Sunnyvale, Calif., and Amersham, Piscataway, N.J.). If the reaction chamber of the system is not entirely enclosed, the reaction chamber is sealed (with mineral oil or other cover).

In some embodiments, the multi-array system is then placed in a warmed thermal cycler or a warm, humidified chamber overnight to allow the single stranded probe DNA to bind to its complementary single stranded target. (Alternatively, the multi-array system may be loaded while already positioned in the thermal cycler.) The system is then removed and the polynucleotide arrays washed (either in the system or after removal from the system) to remove any nonspecifically bound probe. Blocking buffers useful in the detection procedures are readily available to those in the art. Examples include blocking buffers such as Buffer B from the Mergen HO5 ExpressChip™ DNA Microarray System Kit, or its equivalent. Sandwich detection protocols and reagents useful in amplifying the signal from labeled probe are also well known in the art. Suitable detection reagents include the Detection Reagent included in the Mergen HO5 ExpressChip™ DNA Microarray System Kit, or an equivalent of such a reagent. (The Mergen reagent is a Cy3 labeled protein that is used in conjunction with streptavidin.)

The washed arrays are then typically imaged with a confocal laser scanner or other type of scanner suitable for use with the chosen labeling/detection system. A confocal laser scanner contains two lasers tuned to excite the dye incorporated into the DNA probe and a corresponding filter set to select out excitation emission from the dye (e.g., Cy3 or Cy5). The ability to image two fluorescent signals allows for two different polynucleotide samples to be hybridized and directly compared on the same biomolecule array. This excitation emission signal is recorded via a photomultiplier tube (PMT), digitized, and sent to the computer for later analysis. By examining the intensity of a spot's fluorescence, and the ratio of fluorescence between spots, it possible to determine whether a specific gene is being expressed and the relative expression level of the gene between samples. Other available means for labeling and detecting probes, such as with radioisotopes, enzymes, antibodies, biotin, avidin and like materials known in the art, are within the contemplated means of executing the process. A variety of scanners are available in the art for use in reading polynucleotide microarrays such as the GenePix 4000A (Axon Instruments, Union City, Calif.), Affymetrix 417-418 (Affymetrix/Genetic MicroSystems, Santa Clara, Calif.), ScanArray Series (GSI Lumonics, Billerica, Mass.), and ChipReader (Virtek Vision Corp., Canada).

Methods of performing other types of assays involving biomolecule arrays are also well-known to those of ordinary skill in the art and readily adaptable to use in the multi-array system described herein. For instance, in some embodiments, PCR reactions are run in the systems of the present invention. In addition, in some embodiments, polymerase-mediated amplification approaches to detect sequence variation such as the methods described in U.S. Pat. No. 6,376,191, herein incorporated by reference in its entirety, are performed in the systems described herein.

In another aspect, the invention provides a method of making a multi-array system, comprising the following steps: (a) providing a first substrate having a first surface, wherein a first biomolecule array is immobilized on the first surface; (b) providing a second solid substrate having a second surface, wherein a second biomolecule array is immobilized on the second surface; and (c) fixably positioning the first and second solid substrates using a spacer so as to form a reaction chamber in which the first and second surfaces face each other and are separated by a fluid-receiving space, where the first and second biomolecule arrays are exposed to the fluid-receiving space. In some embodiments, the multi-array system is constructed in such a manner that it can be readily disassembled following use to aid in washing of the biomolecule arrays, detection of moieties bound to the biomolecule arrays, or the like.

### EXAMPLES

### Example 1

### Use of a Double-array System in a Hybridization Assay

Two microarrays, an HO5 ExpressChip™ DNA microarray (Mergen, San Leandro, Calif.) and a custom DNA microarray called “CH1”, were used in a hybridization assay. The oligonucleotides on each of the microarrays were designed using an algorithm that selects the optimal 30 contiguous bases that uniquely match with GenBank's human database, with minimal variation in Tₘ and GC contents, low nucleotide repetition, size restriction (30-mer), and consistent position within the gene sequences (relative to mRNA 3′ end). Each microarray has approximately 12,000 sense oligonucleotides printed on it. The gene list for the HO5 DNA microarray is available at the website www.mergen.com/HO5/HO5finder.asp. All the oligonucleotides on the microarrays are 30 bases long with a hydrocarbon spacer (to help avoid steric hindrance). The spacer has an amino group at its distal end to allow for covalent binding of the oligonucleotide to the polymer coated slides.

To make each of the microarrays, the oligonucleotides were contact printed onto polymer-coated glass slides purchased from Surmodics (# DN01-0025, Eden Prairie, Minn., product) or Amersham Biosciences Corp. (Codelink™ Activated Slides, product # 300011, Piscataway, N.J.) in an area approximately 20 mm by approximately 37 mm using a standard printing machine. The arrays were processed as instructed by the slide supplier, Amersham Biosciences. Specifically, the oligonucleotides were printed onto the slides at a concentration of about 0.1-0.5 mg/ml in a printing buffer of 50 mM sodium phosphate, pH 8.5. (The printing were done in an environment where the relative humidity was below 50%.) The slides were then incubated in a saturated NaCl chamber for 4-72 hours at room temperature. Residual reactive groups on the still wet slides were blocked using pre-warmed blocking solution (0.1 M Tris, 50 mM ethanolamine, pH 9.0) at 50° C. for 30 minutes. The slides are then rinsed twice with deionized water and then washed with 4×SSC, 0.1% SDS (pre-warmed to 50° C.) for 30 minutes on a shaker. The slides were then rinsed again with deionized water and spun in a centrifuge at 800 rpm for 3 minutes.

A slide on which the HO5 microarray had been printed and a slide on which the CH1 microarray had been printed were used together to construct a double-array system for the hybridization assay. The two slides were oriented so that the HO5 microarray and the CH1 microarray were facing each other. A thin, soft rubber septum (#SA500, Grace Bio-Labs, Inc., Bend, Oreg.) was placed between the two slides to produce the “sandwiched” hybridization chamber. The volume of the “sandwiched” hybridization chamber was approximately 500 microliters.

These sandwiched microarrays were used to study differential gene expression in various tumor samples and normal control samples from adjacent tissue. Procedures used were essentially identical to those outlined in the manual for the HO5 ExpressChip™ DNA Microarray System (#HO5-001, Mergen, San Leandro, Calif.). Target polynucleotides were prepared from total RNA. First the RNA was reverse transcribed using a primer containing a T7 RNA polymerase promoter. Second strand DNA synthesis was then performed. The cDNA was then transcribed in vitro using the T7 promoter-mediated expression (see, e.g., Luo et al. (1999) Nature Med. 5:117-122) and biotin-rCTP to produce biotinylated antisense RNA probe. The biotinylated RNA probe was then fragmented and denatured into fragments of 30-100 bases. The fragmented and denatured probe was mixed with the hybridization buffer provided in the kit accompanying the HO5 ExpressChip™ DNA Microarray (#HO5-001, Mergen, San Leandro, Calif.) and hybridized to the immobilized oligonucleotides in the “sandwiched” hybridization chamber made of the two arrays. Specifically, a hypodermic needle was used to inject the RNA probe into the hybridization buffer through the septum to fill the hybridization chamber with the hybridization mixture. (A “relief port” was made with another needle at the opposite end of the array to allow escape of air during the filling.)

Hybridization occurred overnight at 42° C. in a rotating, temperature-controlled rotisserie under conditions of high stringency.

After hybridization, the microarrays were washed several times at increasing stringencies to remove unbound probe. The washing procedures were essentially the same as those outlined in the manual for the HO5 ExpressChip™ DNA Microarray System. More specifically, the sandwiched double-array system was disassembled and the slides rinsed briefly by dipping each of the slides into a 50 ml conical tube filled with 2×SSC+0.1% SDS (0.3 M Sodium Chloride, 0.03 M Sodium Citrate, pH 7.0, 0.1% sodium dodecyl sulfate). Next, each slide was washed twice for 10 minutes at a time, with 60 rpm constant shaking, in a solution of 2×SSC+0.1% SDS that had been preheated to 42° C. The slides were then washed in 0.2×SSC (preheated to 37° C.) for 10 min with 60 rpm constant shaking and then in 0.1×SSC for 10 min with 60 rpm constant shaking.

Following the washing steps, the detection procedures were immediately (without allowing the slides to dry) carried out on the remaining, hybridized probe. The detection procedures were carried out in accordance with the instructions provided in the manual for the HO5 ExpressChip™ DNA Microarray System. The reagents used for the detection procedures were identical to those which are provided in the kit accompanying the HO5 ExpressChip™ DNA Microarray, such as blocking buffer, concentrated streptavidin solution, and the detection reagent. The steps used for the detection procedure were the same as those disclosed in the accompanying manual for the HO5 ExpressChip™ DNA Microarray. In general, the remaining, hybridized probe was detected via a “sandwich method” to increase the signal generated by the fluorescent label. (These types of indirect immunofluorescence techniques are known in the art.)

In this example, each of the microarrays was initially incubated for 30 minutes at room temperature with the blocking buffer provided in the kit accompanying the HO5 ExpressChip™ DNA Microarray with constant rotation on an orbital shaker set at 50 rpm. Excess liquid was then tapped off. The microarrays were then incubated with a solution containing streptavidin in the blocking buffer at room temperature or 4° C. for 30 minutes with constant rotation on an orbital shaker. The excess liquid was again tapped off and each slide was placed in a 50 ml conical tube containing 25 ml of a wash buffer. The slides were agitated on an orbital shaker for 15 minutes at room temperature. The step in the conical tube was repeated two more times. The microarrays were then incubated with a detection reagent from the HO5 ExpressChip™ DNA Microarray kit (a Cy3-labelled protein) at room temperature or 4° C. for 30 minutes while shaking. The slides were then again washed three times with the 25 ml of wash buffer as indicated before. The slides were finally rinsed in water and dried.

Images of the microarrays were collected using a commercially available array scanner (#00-0003, GMS 418 scanner, Affymetrix/Genetic MicroSystems, Inc., Santa Clara, Calif.). This laser scanner is compatible with the cyanine-3 fluorescent dye (Cy3) conjugated to the detection reagent.

Table 1 and Table 2, below, show representative data obtained from the microarrays HO5 and CH1, respectively. The data shown (signal after subtraction of local background) is the hybridization data for samples from three patients (Patient #1, Patient #2 and Patient #3) of normal adjacent tissue and the tumor (breast cancer). Therefore, this data set is a representation of the results from the hybridizations of six different samples (normal and healthy tissue from three patients) on a total of 12 arrays (HO5 and CH1 for each hybridization). Only a tiny fraction of the data is shown including representative data for “control genes” (genes not expected to change in cancer), genes not upregulated in cancer, and genes upregulated in cancer.

The two positive control genes found on both the HO5 and CH1 arrays (the genes encoding glyceraldehyde-3-phosphate dehydrogenase (GAPD) and ubiquitin-conjugating enzyme (UBC)) were found to have nearly identical signals because they had been hybridized to the same sample, under the same conditions, in the double-array hybridization system. The positive control genes produced the same signal intensity for both the normal and cancer samples on both types of microarrays for all three patients, as expected. In addition, for some genes tested (identified in the Table 1 as Genes I-L), no difference in the expression level between the normal and cancer tissue samples was observed . Conversely, the cancerous genes (identified here only as Genes A-H) show strong differential expression between the two sample types (normal versus cancer).

TABLE 1
Hybridization data from the HO5 microarray

| HO5 MICROARRAY | | Patient #1 | | Patient #2 | | Patient #3 | |
|---|---|---|---|---|---|---|---|
| GenBank ID | name | Normal | Cancer | Normal | Cancer | Normal | Cancer |
| Controls | | | | | | | |
| M33197 | GAPD | 65536.0 | 65181.2 | 65536.0 | 65219.2 | 65536.0 | 65147.5 |
| M26880 | BC | 65536.0 | 62747.2 | 60607.6 | 64987.6 | 65536.0 | 65153.1 |
| NC1 | Negative (plant) | 8.7 | 8.3 | 3.6 | 4.3 | 1.0 | 3.7 |
| NC2 | Negative (random) | 36.3 | 10.4 | 8.9 | 1.0 | 11.9 | 1.0 |
| Genes NOT upregulated in | | | | | | | |
| cancers | | | | | | | |
| — | Gene I | 1042.8 | 572.4 | 1188.4 | 1224.0 | 1605.5 | 1078.1 |
| — | Gene J | 274.6 | 453.4 | 314.8 | 323.7 | 568.7 | 813.5 |
| — | Gene K | 527.0 | 533.3 | 945.3 | 1448.0 | 526.8 | 4824.4 |
| — | Gene L | 706.5 | 412.1 | 1058.9 | 295.9 | 2358.6 | 370.5 |
| Genes upregulated in cancers | | | | | | | |
| — | Gene A | 1709.6 | 6666.2 | 2064.9 | 4322.8 | 1230.5 | 29391.3 |
| — | Gene B | 215.2 | 1695.6 | 764.8 | 774.5 | 467.3 | 2961.8 |
| — | Gene C | 818.8 | 10746.6 | 1080.1 | 4329.6 | 3919.2 | 6118.3 |
| — | Gene D | 779.0 | 3076.4 | 699.3 | 1234.8 | 638.5 | 4124.3 |

TABLE 2
Hybridization data from the CH1 microarray

| CH1 MICROARRAY | | Patient #1 | | Patient #2 | | Patient #3 | |
|---|---|---|---|---|---|---|---|
| GenBank ID | name | Normal | Cancer | Normal | Cancer | Normal | Cancer |
| Controls | | | | | | | |
| M33197 | GAPD | 65536 | 65346 | 65536 | 62536 | 65536 | 65154 |
| M26880 | UBC | 65536 | 65189 | 65536 | 65278 | 65536 | 65273 |
| K00558 | K-ALPHA-1 | 41457 | 65249 | 54362 | 65122 | 65536 | 64744 |
| M86400 | YWHAZ | 36400 | 65293 | 54886 | 63724 | 65536 | 65156 |
| NC1 | Negative (plant) | 1 | 1 | 18 | 25 | 1 | 1 |
| NC2 | Negative (random) | 5 | 10 | 1 | 23 | 133 | 25 |
| Genes upregulated in cancers | | | | | | | |
| — | Gene E | 5770 | 4268 | 3141 | 14219 | 2392 | 10520 |
| — | Gene F | 207 | 4311 | 1431 | 5814 | 363 | 17012 |
| — | Gene G | 5353 | 20197 | 25654 | 64865 | 7412 | 63341 |
| — | Gene H | 182.3 | 581.1 | 292.2 | 1128.7 | 342.0 | 3073.1 |

The foregoing description of selected embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention.

All publications, patents, and patent applications mentioned in this specification are incorporated herein by reference to the same extent as if each individual publication or patent application were specifically and individually indicated to be incorporated by reference.

What is claimed is:

1. A multi-array system, comprising:
a first solid substrate having a first surface;
a second solid substrate having a second surface, wherein the first and second solid substrates are positioned so that the first surface faces the second surface;
a spacer contacting and separating the first and second solid substrates, so as to form at least one reaction chamber comprising a fluid-receiving space between the first and second surfaces,
a first biomolecule array immobilized on the first surface; and
a second biomolecule array immobilized on the second surface, wherein the first and second biomolecule arrays are exposed to the fluid-receiving space.

2. The multi-array system of claim 1, wherein the reaction chamber is substantially enclosed by the first and second surfaces and the spacer.

3. The multi-array system of claim 1, wherein the reaction chamber comprises one or more openings.

4. The multi-array system of claim 3, wherein the one or more openings are sealable.

5. The multi-array system of claim 1, wherein each of the first and second surfaces is substantially planar.

6. The multi-array system of claim 1, wherein each of the first and second solid substrates comprises a material selected from the group consisting of glass, plastic, and silicon.

7. The multi-array system of claim 6, wherein each of the first and second solid substrates is a coated glass slide or a nylon-overlaid glass slide.

8. The multi-array system of claim 1, wherein the first and second surfaces are in substantially parallel planes.

9. The multi-array system of claim 1, wherein both the first and second biomolecule arrays are polynucleotide arrays.

10. The multi-array system of claim 1, wherein both the first and second biomolecule arrays are protein arrays.

11. The multi-array system of claim 10, wherein both the first and second biomolecule arrays are antibody arrays.

12. The multi-array system of claim 1, wherein the first biomolecule array comprises a different set of biomolecules than the second biomolecule array.

13. The multi-array system of claim 1, wherein the first biomolecule array and the second biomolecule array are substantially identical.

14. The multi-array system of claim 1, wherein the spacer is removably adhered to the first and second surfaces.

15. The multi-array system of claim 1, wherein the spacer forms a watertight seal with the first and second surfaces.

16. The multi-array system of claim 1, wherein the spacer is made of plastic, rubber, or Teflon®.

17. The multi-array system of claim 1, wherein the spacer is made of rubber.

18. The multi-array system of claim 17, wherein the spacer is made of silicone rubber.

19. The multi-array system of claim 17, wherein the spacer is a rubber gasket and the reaction chamber is enclosed by the first surface of the first solid substrate, the second surface of the second solid substrate, and the spacer.

20. The multi-array system of claim 19, wherein the spacer is between about 0.1 mm and about 3 mm thick.

21. The multi-array system of claim 1, wherein the average distance in the reaction chamber between the first surface of the first solid substrate and the second surface of the second solid substrate is between about 0.01 mm and about 2 cm.

22. The multi-array system of claim 21, wherein the average distance is between about 0.01 mm and about 5 mm.

23. The multi-array system of claim 22, wherein the average distance is between about 0.1 mm and about 3 mm.

24. The multi-array system of claim 1, wherein the volume of the reaction chamber is between about 5 μl and about 10 ml.

25. The multi-array system of claim 24, wherein the volume is between about 50 μl and about 1 ml.

26. An apparatus, comprising:
the multi-array system of claim 1; and
a temperature control unit, wherein the temperature control unit functions to alter the temperature of the reaction chamber.

27. The apparatus of claim 26, wherein the temperature control unit is a thermal cycler, a water bath, or an air oven.

28. A method of performing an assay on a plurality of biomolecule arrays simultaneously, comprising using the multi-array system of claim 1 in at least one step of the assay.

29. A method of making a multi-array system, comprising the steps of:
providing a first substrate having a first surface, wherein a first biomolecule array is immobilized on the first surface;
providing a second solid substrate having a second surface, wherein a second biomolecule array is immobilized on the second surface; and
fixably positioning the first and second solid substrates using a spacer so as to form a reaction chamber in which the first and second surfaces face each other and are separated by a fluid-receiving space, and in which the first and second biomolecule arrays are exposed to the fluid-receiving space.

30. A kit, comprising the multi-array system of claim 1.

31. The kit of claim 30, further comprising instructions regarding the use of the multi-array system in at least one step of an assay.
